(19) United States
(12) Patent Application Publication
(10) Pub. No.: US 2006/0229354 A1
(43) Pub. Date: Oct. 12, 2006

(54) Use of Isatin in stimulating red blood cell production and treatment of anemia

(76) Inventor: Honghao Yang, Rancho Palos Verdes, CA (US)

Correspondence Address: Raymond Y. Chan, 108 N. Ynez Ave., #128, Monterey Park, CA, 91754, US

(21) Appl. No.: 11/101,930

(22) Filed: Apr. 7, 2005

(51) Int. Cl. A61K 31/404

(52) U.S. Cl. 514/414; 514/418

(57) ABSTRACT

The present invention concerns small chemical compounds—Isatin and its derivatives, and a novel, safer and more convenient methods for the treatment of anemia resulting from various underlying conditions or diseases. The invention provides new methods to stimulate red cell production, and the new methods to prevent the development of anemia. The invention provides a new therapeutic approach for the treatment of chronic anemia patients, anemia patients who are receiving bone marrow suppressive therapies, HIV/AIDS patients who develop anemia upon the treatment of anti-retroviral therapies or from underlying disease itself, and anemia with other causes. The invention also provides methods for preventing anemia in many relatively healthy people such as young women who loss blood from menstrual blood.

### BACKGROUND OF THE PRESENT INVENTION

1. Field of Invention

The present invention relates to a chemical compounds, Isatin (EA1H-Indole-2,3-dione; 2,3-Indolinedione; Indole-2,3-dione), and its derivatives, and more particularly relates to the method of using Isatin which possess stimulatory effect on red blood cell production for prevention and treatment of anemia. The Isatin and its derivatives can potentially be used for an oral drug.

2. Description of Related Arts

1. The Biology of Red Blood Cell Production:

Red blood cells are generated in the fetal liver and adult bone marrow. This process is regulated by the hormone erythropoietin (EPO). EPO is essential for red blood cell development. EPO binds to a high affinity receptor on the surface of erythroid progenitor cells, stimulating receptor dimerization and activation of the intracellular signal transduction pathways that support erythroid cell survival, proliferation and differentiation.

EPO is a glycoprotein. In adults, almost 90% of EPO is produced in the kidney with the remainder produced by the liver. Renal tubular cells serve as oxygen sensors transmitting signals to EPO-producing cells. These cells activate as the hematocrit drops and secret EPO to stimulate the production of red blood cells in bone marrows. The secretion and activity of EPO is linked in a negative feedback loop, which maintains optimal red cell mass for oxygen transport. There appears to be a plateau of optimal oxygen transport to tissues occurring around hematocrits of 35-55% with significant decreases in oxygen transport above and below these values.

2. Signal Transduction Events Stimulated by EPO:

One of the early signaling events induced by EPO receptor (EPO-R) dimerization after EPO binds to EPO-R is the phosphorylation of cellular proteins on tyrosine residues. This phosphorylation is predominately mediated through JAK pathway, although other tyrosine kinases may also be involved. Eight tyrosine residues are present in the cytoplasmic portion of the EPO-R and several of these have been identified as docking sites for signaling molecules such as STAT5, PI3K p85, and SHP-1. Recruitment of these molecules to the EPO-R complex leads to the activation of several signaling cascades, resulting in the changes in subcellular localization, tyrosine phosphorylation, and/or oligomerization. In addition, tyrosine phosphorylated JAK2 may directly interact with certain signaling molecules, including STAT5 and members of CIS/JAB/SOCS family of cytokine inhibitory proteins.

3. Survival and Apoptosis (Program Cell Death) of Hematopoietic Progenitor Cells:

Survival and apoptosis of hematopoietic progenitor cells are crucial in maintaining the homeostasis of blood cell production, and hematopoietic growth factors are crucial for controlling the balance between survival and apoptosis. Apoptosis can be initiated by withdrawal of growth factors, such as EPO. The apoptotic pathway involves multiple components and one of the central elements of the pathway is the Bcl-2 family of proteins. Growth factors and other survival factors inhibit apoptosis by up-regulating the expression of the antiapoptotic Bcl-2 family proteins and by inactivating proapoptotic family proteins.

4. Epoetin Alfa Used for the Treatment of Anemia:

Defects in EPO production lead to severe anemia due to the absence of circulating red blood cells. Since the major site of EPO synthesis is the kidney in the adult, patients with renal failure require supplemental EPO to maintain normal levels of red blood cells. Bone marrows suppressive therapies used to treat cancer or AIDS can also reduce circulating red blood cell levels and lead to anemia. EPO treatment such as epoetin alfa has been used to correct the anemia.

Development of the recombinant epoetin alfa was begun in 1983 following the discovery of the EPO gene on chromosome 7. Epoetin alfa was developed by Amgen, Inc. and approved by FDA for the treatment of anemia. Epoetin alfa produces a dose-dependent increase in the hematocrit; an increase of 2% per week may be seen during the initial phase of therapy.

### SUMMARY OF THE PRESENT INVENTION

The main object of the present invention is to provide a novel method of anemia treatment by administering Isatin and/or its derivatives, wherein the Isatin has a chemical formula EA1H-Indole-2,3-dione; 2,3-Indolinedione; Indole-2,3-dione and possesses stimulatory effect on red blood cell production thereby can be used for anemia prevention and treatment.

The second object of the present invention is to provide a novel way to treat anemiaby formulating Isatin and/or its derivatives as such that they can potentially be used for oral drugs for the purpose of anemia treatment and prevention.

The third object of the present invention is to provide evidences in supporting the fact that Isatin and/or its derivatives use similar mechanism as erythropoietin by activating EPO receptor signaling pathway and thereby stimulating the red blood cell production from bone marrow. The forth object of the present invention is to provide evidences demonstrating that Isatin (and/its derivatives) and erythropoietin are synergistic in stimulating red cell production from bone marrow. Therefore, Isatin and/or its derivatives can be applied together in treating and preventing anemia. In order to accomplish the above objects, the present invention provide a method of treatment of anemia, comprising the step of (a) administering a predetermined quantity of Isatin, wherein the Isatin have a chemical formulae EA1H-Indole-2,3-dione; 2,3-Indolinedione; Indole-2,3-dione. The Isatin has stimulatory effect on red blood cell production.

Furthermore, the Isatin and/or its derivatives has a similar mechanism of action as erythroprotein in stimulating bone marrow red blood cell production. The Isatin have a synergistic effect on stimulating red cell production from bone marrow. The derivatives of Isatin includes D-1, D-2, D-3, D-4, D-5 and D-6.

These and other objectives, features, and advantages of the present invention will become apparent from the following detailed description, the accompanying drawings, and the appended claims.

### BRIEF DESCRIPTION OF THE DRAWINGS

FIG. 1 illustrates the chemical structures of Isatin and its derivatives (D-1 to D-6) of the present invention.

FIG. 2A illustrates the results of AA studies and shows that the increase in Isatin concentration correlates with the decrease in apoptosis of UT7/EPO cells.

FIG. 2B illustrates the viability of UT7/EPO cells measured by calcein assay.

FIG. 3A illustrates the synergistic effect in inhibiting apoptosis of UT7/EPO cells between Isatin and EPO, wherein the results of AA for the measurement of the apoptosis in UT7/EPO cells are shown.

FIG. 3B illustrates the synergistic effect in inhibiting apoptosis of UT7/EPO cells between Isatin and EPO, wherein the results of calcein assay for the measurement of the viability of UT7/EPO cells are shown.

FIG. 4 illustrates the results of calcein assay for the measurement of the viability of EPO-deprived UT7/EPO and GM-CSF-deprived MO7E cells in response to Isatin treatment.

FIG. 5A illustrates the results of immunoprecipiate studies for the assessment of the EPO and Isatin effects on phosphorylation of JAK2-associated EPOR in UT7/EPO Cells, which shows that Isatin induces phosphorylation of JAK2-associated EPOR.

FIG. 5B illustrates the results of immunoprecipiate studies for the assessment of the EPO and Isatin effects on phosphorylation of JAK2-associated EPOR in UT7/EPO Cells, which shows that Isatin-induced EPOR phosphorylation occurs 5 minutes after treatment.

FIG. 6 illustrates the results of Immunoprecipiate analysis of the effects of EPO and Isatin on STAT5 phosphorylation in UT7/EPO Cells.

### DETAILED DESCRIPTION OF THE PREFERRED EMBODIMENT

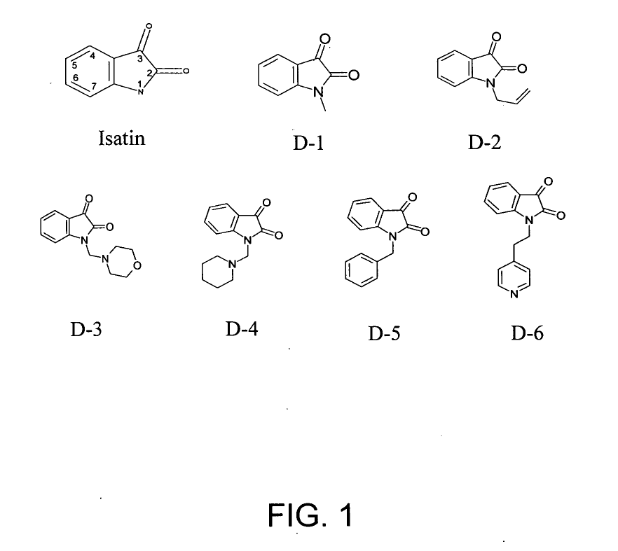

Referring to FIG. 1 of the drawings, the present invention concerns a new approach for the treatment of anemia using novel chemical compounds, Isatin (EA1H-Indole-2,3-dione; 2,3-Indolinedione; Indole-2,3-dione), and its derivatives. Isatin and its derivatives represent the first example of chemical compounds that possess stimulatory effect on red blood cell production. Isatin and its derivatives can potentially be used as an oral drug for the prevention and treatment of anemia.

In FIG. 1, the chemical structure of Isatin and its derivatives, D-1, D-2, D-3, D-4, and D-5 are demonstrated.

The present invention provides a novel biologically active compound Isatin and its derivatives that stimulate the production of red blood cells through its inhibition on red cell apoptosis, a program cell death process that plays a major role in regulating red cell number in the blood. The invention provides a foundation of a new approach in anemia treatment by using small molecules such as Isatin and its derivatives as oral drugs to stimulate the production of red blood cells from human bone marrows.

Development of Oral Erythropoietin Mimetics

Epoetin alfa is a recombinant protein, epoetin alfa cannot be taken orally. We decided to develop a new generation of hematopoietic stimulants by using small molecules. We have researched hundreds of volumes of literatures in Chinese herbal medicine and analyzed the chemical components in various Chinese medicinal herbs that have been shown to be effective in treating patients with anemia. We eventually found a class of small chemical compounds, Isatin and its derivatives (FIG. 1), that are active in stimulating red blood cell production. The small chemical molecules of this invention cover Isatin, the derivatives of Isatin demonstrated in the FIG. 1, all the members of chemical compounds that belong to the same family of Isatin in chemical structures, and all the other chemical compounds whose structures are similar or derived from the basic chemical structure of Isatin.

Materials and Methods

1. Cell culture: UT7/EPO cells were obtained from ATCC

2. Assays to measure cell viability and apoptosis: Apoptosis Assay (AA), DNA Ladder Detection Assay, MTT Assay and Calcein-AM Release Assay are used.

Apoptosis Assay (AA)

UT7/EPO cells treated by Isatin or EPO or PBS was plated in triplicate in 96-well microtiter plates in 50 μl of culture medium and incubated for different time course. The cell apoptosis products were determined by Florence-probe. The cell apoptosis rate was calculated by positive apoptosis fragments.

DNA Ladder Detection Assay

10⁶ UT7/EPO cells, after treated with either Isatin or EPO or PBS, was lysed with 150 ul hypotonic lysis buffer (10 mM EDTA, 0.5% Triton X-100 in 0.05 mM Tris-HCl, pH 7.4) for 15 minutes on ice and were precipitated with 2.5% polyethylene glycerol and 1 M NaCl for 15 minutes at 4 C. After centrifugation at 16,000 rpm for 10 minutes at room temperature, the supernatant was incubated in the presence of proteinase K (0.3 g/L) at 37° C. for one hour and precipitated with isopropanol at −20° C. After centrifugation, each cell pellet was dissolved in 10 ul of Tris-EDTA (pH 7.6) and electrophoresed on a 1.5% agarose gell containing ethidium bromide. Ladder formation of oligonucleosomal DNA was detected under ultraviolet.

MTT Assay

UT7/EPO cells were cultured for 4 hours and then placed in triplicate in 96 well microtiter plates. The cell was treated with Isatin or EPO or PBS and cultured in 100 ul of DMEM, 10% FCS, overnight. The next day, media was removed and replace MTT assay solution. The plates were incubated for and addition 48 hours. And then cell viability was determined using a MTT assay, as described by manufacture (Sigma).

Calcein-AM Release Assay

Calcein-AM was purchased from Molecular Probes (Eugene) as a 1 mg/ml solution in dry dimethyl sulfoxide. UT7/EPO cells were resuspended in complete medium at a final concentration of 10⁶/ml and incubated with 15 uM calcein-AM for 30 mininutes at 37° C. with occasional shaking. After two washes in the same way as the ⁵¹Cr assay in V bottom 96-well microtiter plates, with E:T ratios ranging from 50:1 to 0.5:1, in triplicate, and with at least six replicate wells for spontaneous and maximum release. Various numbers of UT7/EPO cells treated with Isatin or EPO or with untreated cells were seeded as follows. After incubation 37° C. in 5% CO₂ for 4 hours, 75 ul of each supernatant was harvested and transferred into new plates. The samples were measured using a Sepctramax Gemini dual-scanning microplate spectrofluoimeter. Data were expressed as arbitrary fluorescent unites (AFU). Percent lysis was calculated with the same formula used for the ⁵¹Cr for the ⁵¹CR assay.

The Brief Description of the Results are Summarized as Follows:

1. Demonstration of Erythropoietic Stimulatory Effect of Isatin

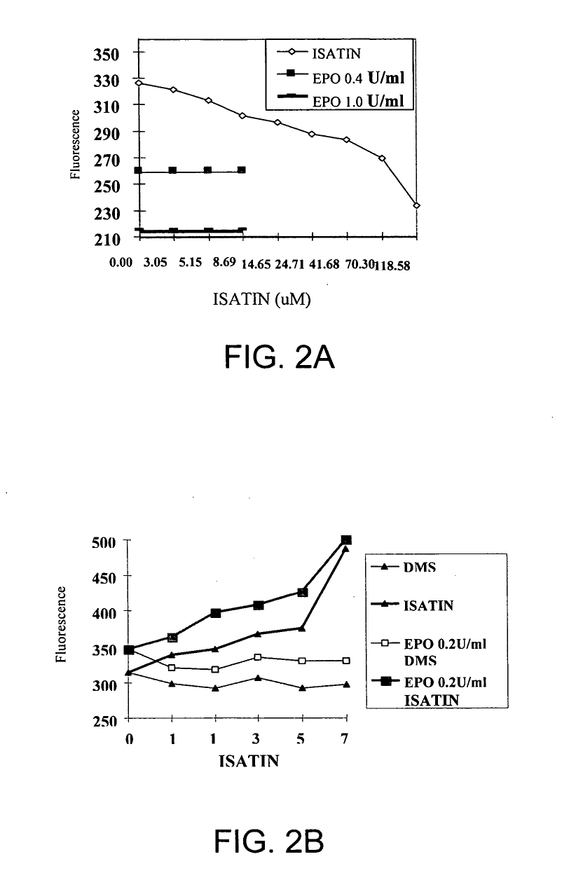

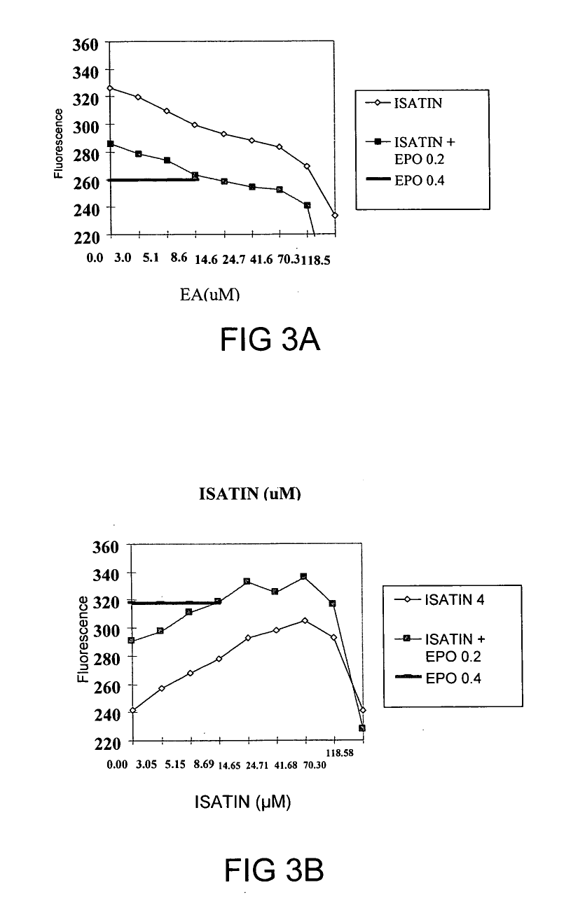

We used several assays as mentioned in Materials and Methods section to evaluate the pharmacological activity of Isatin compound by examining the growth or the apoptosis of UT7/EPO cells. The cells used in AA assay are UT7/EPO. UT7/EPO cells were deprived from UT7 by elegant genetic engineering. As a result, the survival and growth of UT7/EPO cells rely on the stimulation of EPO-R by the exogenous EPO treatment. On the other hand, without the support of the exogenous EPO, the UT7/EPO cells will die. This can be demonstrated by the increase in apoptosis of UT7/EPO cells. We used different assays to measure the level of apoptosis in UT7/EPO cells. If the tested compounds have erythropoietic agonist effect, the level of apoptosis in UT7/EPO cells should decrease in response to the treatment. The apoptosis inhibition on UT7/EPO cells was steadily enhanced when the concentration of Isatin was increased, as shown in FIGS. 2A and 2B. Compared to EPO, higher dose of Isatin was required to generate the same extent of inhibition. However, if used together with EPO even at lower dose, Isatin has similar effect as that can be achieved by EPO alone at higher dose, as shown in FIG. 3, suggesting a synergistic effect between EPO and Isatin on erythropoietic stimulation.

In conclusion, Isatin has been proven to be biologically active and stimulate the proliferation of UT7/EPO cells. Isatin's effect appears to be weaker than that of EPO and its IC50 for inhibiting apoptosis in UT7/EPO cells is around 10 μM. There is a synergistic effect between Isatin and EPO in stimulating EPO-R activity.

2. Specificity Studies on the Erythropoietic Stimulatory Effect of Isatin

The inhibition of apoptosis by Isatin is only observed in UT7/EPO cells. The data is summarized in Table 1.

TABLE 1
Comparison of the effects of Isatin on apoptosis in the EPO-deprived
UT7/EPO and GM-CSF-deprived MO7E cells.

| | EPO-Deprived | GM-CSF-Deprived |
|---|---|---|
| Isatin Treatment | UT7/EPO Cells | MO7E Cells |
| AA | ↓ | — |
| MTT | ↑ | — |
| Calcein | ↑ | N.D. |
| DNA Ladder Assay | ↓ | N.D. |
| Viable Cell Counts | ↑ | — |
| (trypan blue staining) | | |

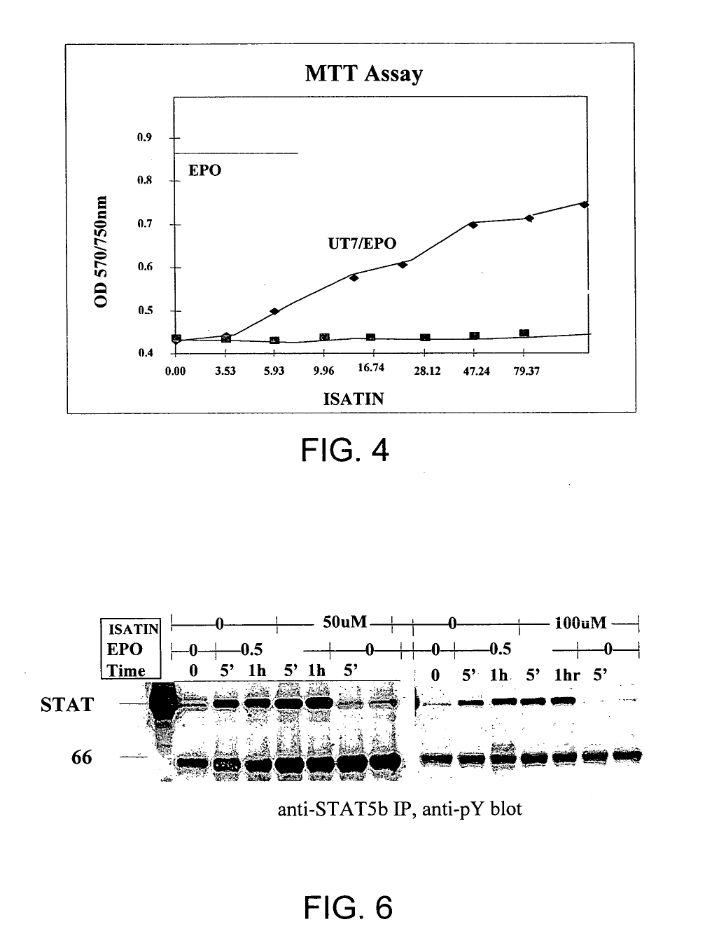

Referring to Table 1, there is no noted inhibition on (a) Melphalan-induced apoptosis in RPM18226 cells (data not shown); (b) Melphalan-induced apoptosis in freshly isolated peripheral blood mononuclear cells (data not shown); and GM-CSF withdrawal-induced apoptosis in MO7E cells, as shown in FIG. 4.

Therefore, we concluded that the effect of Isatin is very specific for the EPO-R. It only stimulates the cell growth that is mediated by EPO-R activation. It does not activate other cytokine or growth factor receptors. In this regard, Isatin mimics the biological activity of EPO.

3. Mechanism of Action

After demonstrated its erythropoietic stimulatory activity, we then examined the molecular mechanism that underlies Isatin functions.

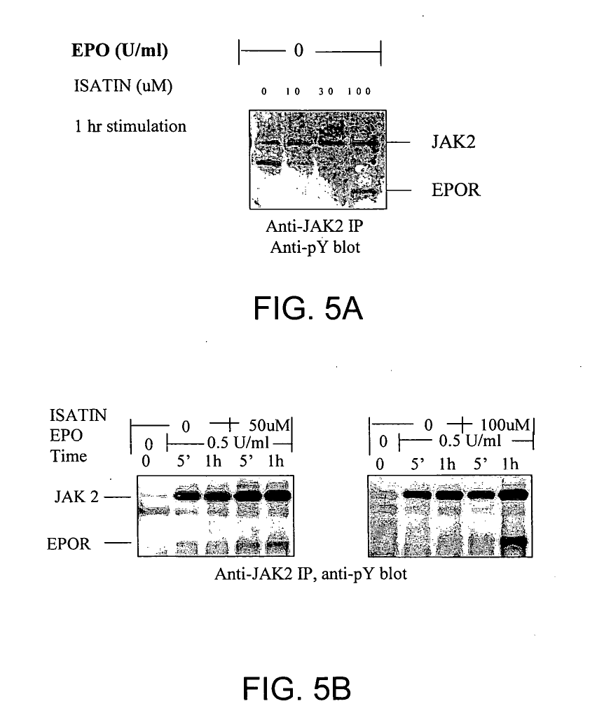

Because the apoptosis inhibitory effect of Isatin compound is specific to UT7/EPO cells, we believe that the mechanisms of action of Isatin are most likely through activation of EPO-R. As mentioned earlier, one of the consequences of EPO-R stimulation is to phosphorylate EPO-R. Therefore, we set forth to examine the phosphorylation of EPO-R. As expected, Isatin induces phosphorylation of EPO-R through JAK2 mediated signaling pathway as demonstrated by immunoprecipitate studies, as shown in FIGS. 5A and 5B. Referring to FIG. 5B, the Isatin-induced EPO-R phosphorylation occurs 5 minutes after the treatment and sustains beyond 1 hour afterwards. EPO can also induced JAK2-associated EPO-R phosphorylation. However, EPO's activity is much weaker than that of Isatin. In addition, when Isatin is used together with EPO, they significantly enhance the phosphorylation of EPO-R.

Activation of EPO-R by EPO is predominately mediated by JAK signaling pathway, and STAT5 protein is the major component involving in this signal transduction. We studied the activity of STAT5 in response to Isatin treatment. Unlike EPO, to our surprise, there was no change in STAT5 activity during the Isatin treatment, as shown in FIG. 6.

4. Study Summary

Isatin has similar erythropoietic stimulatory effects as EPO, demonstrated by inhibition of apoptosis in UT7/EPO cells.

There is a great synergy between EPO and Isatin in stimulating red cell production. When Isatin was used together with EPO, only small amount of EPO was required to achieve similar stimulatory effect as those induced by EPO alone at higher doses.

The erythropoitic stimulatory effect of Isatin is specific. There is no noted effect on the growth and proliferation of other cells (their survival does not depend on EPO stimulation).

The molecular mechanisms that underlie the stimulatory effects of Isatin on erythropoiesis are not clear.

The effects of Isatin are at least partially mediated by EPO-R. However, the exact ways to stimulate EPOR by Isatin are different from those of EPO. The different mechanisms of action between Isatin and EPO are probably the basis of synergy between the two drugs in stimulating RBC production.

JAK pathway is believed to participate in the regulation process induced by EPO. The Isatin's effects also appear to be mediated by JAK signaling. This is supported by the fact that Isatin induces JAK-associated EPOR phosphorylation. However, the exact signaling pathway induced by Isatin is not very clear. Unlike EPO, EOPR phosphorylation induced by Isatin does not lead to the phosphorylation of STAT5, an important protein in JAK signaling pathway.

The Therapeutic Uses of the Small Chemical Molecules of the Invention—Isatin and it derivatives are studied.

The present invention provides new therapeutic strategies for treating anemia diseases resulting from either excess blood loss or inappropriate bone marrow red cell production. The invention provides advantages over existing methods for treating anemia diseases. The methods described herein find use in the treatment of any mammalian subject, however, humans are a preferred subject.

Nature of the Diseases Targeted:

Millions of people suffer from anemia with different causes. It is one of the most common diseases and the majority of anemia patients can benefit from hematopoietic stimulating treatment. The need for anemia treatment is tremendous. Isatin and its derivative small chemical compounds represent the first drug in its class that is developed for using as an oral drug to prevent and treat anemia. Essentially, all the anemic patients currently receiving EPO injections will benefit from an oral anemia drug such as Isatin and its derivatives. In addition, many more patients who have only mild anemia and require no EPO injection can benefit from Isatin and its derivatives. Most anemic patients contain endogenous EPO in their blood. They may only need to take oral drug like Isatin to maintain their normal hemoglobin level because of the synergy between Isatin and EPO in promoting red blood cell production.

As a potential oral drug for anemia prevention and treatment, the Isatin and its chemical derivative compounds disclosed herein can be used to treat or prevent a large number of anemia diseases as described as follows:

1. Chronic Renal Failure Patients Who Suffer from Anemia

More than 1 million people in the US alone suffer from chronic renal failure and approximately ⅓ of those patients have low red blood cell production. Before the development of epoetin alfa, many of these patients could not maintain vitality without regular blood transfusion. But this estimate just serves as a starting point. The debilitating impact of anemia, which causes fatigue, impaired cognitive and physical functioning, and may contribute to cardiovascular disease, occurs in a number of medical settings. Availability of an oral drug as Isatin will be the second revolutionized advancement in the treatment for anemia. It will make the treatment much easier, thereby improving the patients' quality of life.

2. Cancer Patients Who Receive Chemotherapy and Radiation Therapies

More than 8 million people around the world are diagnosed with cancer each year. In the United States alone, the number exceeds one million. Chemotherapy and radiation remain the most widely chosen treatment options for many types of cancers. Unfortunately, both chemo- and radiotherapies have side effects. Among them, one of the most common ones is anemia. Anemia can either result form cancer-related treatment or the underlying disease itself. Before, many cancer patients required the support of frequent blood transfusion. Since the availability of epoetin alfa, the majority of cancer patients are now receiving epoetin alfa treatment. Convenient oral anemia medication as Isatin and its derivatives can help this group of patients tremendously.

3. HIV/AIDS patients can develop anemia upon the treatment of anti-retroviral therapies or from underlying disease itself. They will also benefit from oral drugs such as Isatin and its derivatives by boosting up their RBC production.

4. Anemia patients with many other causes

5. Anemia prevention in many relatively healthy people such as young women who loss blood from menstrual blood.

The Effect of the Composition of the Present Invention:

As mentioned earlier, Epoetin alfa is an effective drug. However, it is expensive and it is not easy to administer. As a recombinant protein, epoetin alfa has to be administered through subcutaneous or intravenous route. During the administration, to prevent adherence to the tubing, epoetin alfa should be injected while blood is still in the IV line, followed by flushing with normal saline. The storage of the drug is also not very convenient. Epoetin alfa has to be stored in a refrigerator between 2° C. and 8° C. (36 and 46 F). It cannot be frozen. The shelf life of multi-dose vials in refrigerator is only about 20 days after the initial dose. On the other hand, as a small chemical compound, Isatin and its derivatives are much more stable and can potentially be used as oral drugs. In addition, they should not have the drug storage problem that is often associated with recombinant epoetin alfa.

Compositions and Formulations of the Present Invention:

For therapeutic uses, including prevention, the compounds of the invention can be formulated as pharmaceutical compositions in admixture with pharmaceutically acceptable carriers or diluents. A suitable administration format can best be determined by a medical practitioner for each patient individually.

Pharmaceutical compositions of the present invention can comprise a small molecule of the present invention along with conventional carriers and optionally other ingredients.

Suitable forms, in part, depend upon the use or the route of entry, for example oral, transdermal, inhalation, or by injection. Such forms should allow the agent or composition to reach a target cell whether the target cell is present in a multicellular host or in culture. For example, pharmacological agents or compositions injected into the blood stream should be soluble. Other factors are known in the art, and include considerations such as toxicity and forms that prevent the agent or composition from exerting its effect.

Carriers or excipients can also be used to facilitate administration of the compound. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents. The compositions or pharmaceutical composition can be administered by different routes including, but not limited to, oral, intravenous, intra-arterial, intraperitoneal, subcutaneous, intranasal or intrapulmonary routes.

The desired isotonicity of the compositions can be accomplished using sodium chloride or other pharmaceutically acceptable agents such as dextrose, boric acid, sodium tartrate, propylene glycol, polyols (such as mannitol and sorbitol), or other inorganic or organic solutes.

For systemic administration, oral administration is preferred. The compounds are formulated into conventional oral dosage forms such as capsules, tablets and tonics. Alternatively, certain molecules identified in accordance with the present invention can be administered by injection, e.g., intramuscular, intravenous, intra-arterial, etc. For injection, the compounds of the invention are formulated in liquid solutions, preferably in physiologically compatible buffers such as Hank's solution or Ringer's solution. Alternatively, the compounds of the invention are formulated in one or more excipients (e.g., propylene glycol) that are generally accepted as safe as defined by USP standards. They can, for example, be suspended in inert oil, suitably a vegetable oil such as sesame, peanut, olive oil, or other acceptable carrier. Preferably, they are suspended in an aqueous carrier, for example, in an isotonic buffer solution at pH of about 5.6 to 7.4. These compositions can be sterilized by conventional sterilization techniques, or can be sterile filtered. The compositions can contain pharmaceutically acceptable auxiliary substances as required to approximate physiological conditions, such as pH buffering agents. Useful buffers include for example, sodium acetate/acetic acid buffers. A form of repository or “depot” slow release preparation can be used so that therapeutically effective amounts of the preparation are delivered into the bloodstream over many hours or days following transdermal injection or delivery. In addition, the compounds can be formulated in solid form and redissolved or suspended immediately prior to use. Lyophilized forms are also included.

Systemic administration can also be by transmucosal or transdermal. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents can be used to facilitate permeation. Transmucosal administration can be, for example, through nasal sprays or using suppositories.

A preferred route for administration of the compounds of the invention may be inhalation for intranasal and/or intrapulmonary delivery. For administration by inhalation, usually inhalable dry power compositions or aerosol compositions are used, where the size of the particles or droplets is selected to ensure deposition of the active ingredient in the desired part of the respiratory tract, e.g. throat, upper respiratory tract or lungs. Inhalable compositions and devices for their administration are well known in the art. For example, devices for the delivery of aerosol medications for inspiration are known. One such device is a metered dose inhaler that delivers the same dosage of medication to the patient upon each actuation of the device. Metered dose inhalers typically include a canister containing a reservoir of medication and propellant under pressure and a fixed volume metered dose chamber. The canister is inserted into a receptacle in a body or base having a mouthpiece or nosepiece for delivering medication to the patient. The patient uses the device by manually pressing the canister into the body to close a filling valve and capture a metered dose of medication inside the chamber and to open a release valve which releases the captured, fixed volume of medication in the dose chamber to the atmosphere as an aerosol mist. Simultaneously, the patient inhales through the mouthpiece to entrain the mist into the airway. The patient then releases the canister so that the release valve closes and the filling valve opens to refill the dose chamber for the next administration of medication.

Another device is the breath actuated metered dose inhaler that operates to provide automatically a metered dose in response to the patient's inspiratory effort. One style of breath actuated device releases a dose when the inspiratory effort moves a mechanical lever to trigger the release valve. Another style releases the dose when the detected flow rises above a preset threshold, as detected by a hot wire anemometer.

Devices also exist to deliver dry powdered drugs to the patient's airways and to deliver an aerosol by heating a solid aerosol precursor material. These devices typically operate to deliver the drug during the early stages of the patient's inspiration by relying on the patient's inspiratory flow to draw the drug out of the reservoir into the airway or to actuate a heating element to vaporize the solid aerosol precursor.

For topical administration, the compounds of the invention are formulated into ointments, salves, gels, or creams, as is generally known in the art.

If desired, solutions of the above compositions can be thickened with a thickening agent such as methyl cellulose. They can be prepared in emulsified form, either water in oil or oil in water. Any of a wide variety of pharmaceutically acceptable emulsifying agents can be employed including, for example, acacia powder, a non-ionic surfactant (such as a Tween), or an ionic surfactant (such as alkali polyether alcohol sulfates or sulfonates, e.g., a Triton).

Compositions useful in the invention are prepared by mixing the ingredients following generally accepted procedures. For example, the selected components can be mixed simply in a blender or other standard device to produce a concentrated mixture which can then be adjusted to the final concentration and viscosity by the addition of water or thickening agent and possibly a buffer to control pH or an additional solute to control tonicity.

The amounts of various compounds for use in the methods of the invention to be administered can be determined by standard procedures. The determination of the actual dose is well within the skill of an ordinary physician.

The compounds of the present invention may be administered in combination with one or more further therapeutic agent for the treatment of anemic diseases or conditions. Such further therapeutic agents include, without limitation, soluble recombinant proteins such as Epoetin alfa, procrit, aranesp, G-CSF, and GM-CSF, and other medications that are used for anemia treatment such as corticosteroids, iron sulfate, and many others.

### CONCLUSION

The appearance of anemia across such a wide spectrum of medical conditions and treatment settings underscores the broad potential of Isatin and its derivatives—being developed as a preferably oral anemia drug. The need for an oral anemia drug is great.

Compared to epoetin alfa, oral drug Isatin and its derivatives can be more user-friendly. It can substantially simplify the anemia management for patients and health care provides alike.

The synergy between Isatin and EPO provides a new strategy to treat the majority of anemic patients.

Our current studies on Isatin and its chemical derivatives pave the way for future development in drug design by modifying the structure of Isatin and its derivative compounds in order to achieve more effective drugs

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure form such principles. Therefore, this invention includes all modifications encompassed within the spirit and scope of the following claims.

What is claimed is:

1. A method of treatment of anemia, comprising the step of (a) administering a predetermined quantity of Isatin, wherein said Isatin have a chemical formulae EA1H-Indole-2,3-dione; 2,3-Indolinedione; Indole-2,3-dione.

2. The method, as recited in claim 1, wherein said Isatin has stimulatory effect on red blood cell production.

3. The method, as recited in claim 1, wherein said Isatin has similar mechanism of action as erythropoietin in stimulating bone marrow red blood cell production.

4. The method, as recited in claim 3, wherein said Isatin and erythropoietin have a synergistic effect on stimulating red blood cell production from bone marrow.

5. The method, as recited in claim 1, wherein said Isatin is replaced by a plurality of derivatives comprising the group consisting of D-1, D-2, D-3, D-4, D-5 and D-6.

6. The method, as recited in claim 5, wherein said Isatin's derivatives have a stimulatory effect on red blood cell production.

7. The method, as recited in claim 5, wherein said Isatin's derivatives have a similar mechanism of action as erythropoietin in stimulating bone marrow red blood cell production.

8. The method, as recited in claim 5, wherein said Isatin's derivatives and erythropoietin have a synergistic effect on stimulating red blood cell production from bone marrow.

9. The method, as recited in claim 2, wherein said Isatin is combined with an acceptable carrier.

10. The method, as recited in claim 6, wherein said Isatin's derivatives are combined with an acceptable carrier.

11. The method, as recited in claim 2, wherein said Isatin is combined with an excipient.

12. The method, as recited in claim 6, wherein said Isatin's derivatives are combined with an excipient.

13. The method, as recited in claim 11, wherein said excipient is selected from the group consisting of calcium carbonate, calcium phosphate, lactose, glucose, sucrose, starch cellulose derivatives, gelatin, vegetable oils, polyethylene glycols and physiologically compatible solvents.

14. The method, as recited in claim 12, wherein said excipient is selected from the group consisting of calcium carbonate, calcium phosphate, lactose, glucose, sucrose, starch cellulose derivatives, gelatin, vegetable oils, polyethylene glycols and physiologically compatible solvents.

15. The method, as recited in claim 2, wherein in step (a) administration is selected from the group consisting of oral, intravenous, intra-arterial, intraperitoneal, subcutaneous, intranasal and intrapulmonary administration.

16. The method, as recited in claim 6, wherein in step (a) administration is selected from the group consisting of oral, intravenous, intra-arterial, intraperitoneal, subcutaneous, intranasal and intrapulmonary administration.

17. The method, as recited in claim 13, wherein in step (a) administration is selected from the group consisting of oral, intravenous, intra-arterial, intraperitoneal, subcutaneous, intranasal and intrapulmonary administration.

18. The method, as recited in claim 14, wherein in step (a) administration is selected from the group consisting of oral, intravenous, intra-arterial, intraperitoneal, subcutaneous, intranasal and intrapulmonary administration.

19. The method, as recited in claim 2, furthering comprising a step of (b) adjusting an isotonicity of said Isatin using an agent, wherein said agent is selected from the group consisting of sodium chloride, dextrose, boric acid, sodium tartrate, propylene glycol, polyols, mannitol, sorbitol, inorganic solutes and organic solutes.

20. The method, as recited in claim 6, furthering comprising a step of (b) adjusting an isotonicity of said derivative using an agent, wherein said agent is selected from the group consisting of sodium chloride, dextrose, boric acid, sodium tartrate, propylene glycol, polyols, mannitol, sorbitol, inorganic solutes and organic solutes.

21. The method, as recited in claim 13, furthering comprising a step of (b) adjusting an isotonicity of said Isatin using an agent, wherein said agent is selected from the group consisting of sodium chloride, dextrose, boric acid, sodium tartrate, propylene glycol, polyols, mannitol, sorbitol, inorganic solutes and organic solutes.

22. The method, as recited in claim 14, furthering comprising a step of (b) adjusting an isotonicity of said derivative using an agent, wherein said agent is selected from the group consisting of sodium chloride, dextrose, boric acid, sodium tartrate, propylene glycol, polyols, mannitol, sorbitol, inorganic solutes and organic solutes.

23. The method, as recited in claim 1, further comprising a step of (b′) administering a supplementary constitutent together with said Isatin, wherein said supplementary constituent is selected from the group consisting of Epoetin alfa, procrit, aranesp, G-CSF, GM-CSF, corticosteroids and iron sulfate.

24. The method, as recited in claim 5, further comprising a step of (b′) administering a supplementary constitutent together with said derivative, wherein said supplementary constituent is selected from the group consisting of Epoetin alfa, procrit, aranesp, G-CSF, GM-CSF, corticosteroids and iron sulfate.
